(19) United States
(12) Patent Application Publication
(10) Pub. No.: US 2026/0055071 A1
(43) Pub. Date: Feb. 26, 2026

(54) PROCESS FOR PREPARING BETA-LACTONES

(71) Applicant: Novomer, Inc., Rochester, NY (US)

(72) Inventors: Jonathan D. Tedder, Rochester, NY (US); Geoffrey W. Coates, Lansing, NY (US)

(21) Appl. No.: 19/104,317

(22) PCT Filed: Aug. 31, 2023
(86) PCT No.: PCT/US2023/031662

§ 371 Date: Feb. 17, 2025

Related U.S. Application Data
(60) Provisional application No. 63/403,335, filed on Sep. 2, 2022.

(51) Int. Cl. C07D 305/12

(52) U.S. Cl. CPC C07D 305/12

(57) ABSTRACT

Disclosed are methods which comprise the preparation of beta-lactones which provide for shorter reaction times with lower by-product formation. The method comprises contacting carbon monoxide with an epoxide in the presence of a carbonylation catalyst to form a reaction mixture under conditions such that the carbon monoxide distributed throughout the reaction mixture and the reaction mixture is substantially saturated with carbon monoxide. The reaction conditions and reactor designs are chosen to distribute the carbon monoxide throughout the reaction mixture and to maintain the reaction mixture as substantially saturated with carbon monoxide. Under these conditions the formation of beta-lactones over the formation of by-products is favored.

### TECHNICAL FIELD

Disclosed are methods for more efficiently preparing beta-lactones from one or more epoxides and carbon monoxide. The disclosed methods prepare the beta lactones using shorter residence times in reactors wherein the prepared beta lactones exhibit lower levels of impurities.

### BACKGROUND

Carbonylation is a process that can be used to react carbon monoxide and an epoxide to make a lactone. In some cases, additional steps are taken to react the lactones to prepare polymers. These lactones may be used as disinfectants and the polymers thereof can be used as plastics. The lactones can be polymerized, and the polymers can be used to prepare acrylic acid. The carbonylation process can be used to prepare organic compounds such as, succinic acid and cyclic anhydrides. The polymers may be utilized in coatings and to prepare plastic structures which are compostable. When making these lactones, a carbonylation catalyst is used to optimize the efficiency of the reaction to produce lactones at competitive prices. Carbonylation catalysts are expensive. The catalyzed reactions of a gas, such as carbon monoxide, with an epoxide have typically been performed in stirred batch or continuously stirred reactors maintaining an overpressure of the reactant gas and continuous injection of the gas reactant into the liquid. Batch reactors tend to efficiently use the catalyst (i.e., have a high turnover number “TON” of the catalyst), but suffer from high capital costs for given throughput and down time between batches. Continuously stirred reactors (CSTRs) may continuously produce product, but typically require increased loading of catalyst to realize desired productivity, requiring inefficient use of the catalyst. The inefficient use of catalyst is generally overcome by continually separating, recycling and replenishing the catalyst, which undesirably adds complexity and problems such as fouling of separation membranes and the like. One method of increasing the efficiency of a process is to utilize higher temperatures to perform the process to increase catalyst productivity. Unfortunately, the use of higher temperatures may result in the production of higher levels of impurities that potentially decrease catalyst productivity. These by-products could also present issues in distillation, interfere with polymerization, and can make end use approvals more difficult.

The continuous carbonylation of epoxides such as ethylene oxide employing recycling of a catalyst has been described in U.S. Pat. No. 9,493,391 which describes various parameters for performing the reaction and suggests that the catalyst is deactivated at 90° C.

There is a need for processes that prepare lactones that are more efficient so as to lower the cost of the preparation of the lactones wherein the processes prepare lactones with lower levels of impurities.

### SUMMARY

The disclosed method improves the rate of reaction and reduces the formation of impurities. Disclosed is a method comprising contacting carbon monoxide with one or more epoxides in one or more liquid solvents in the presence of one or more carbonylation catalysts to form a reaction mixture in the liquid solvents, feeding gaseous carbon monoxide to the reaction mixture such that the reaction mixture is under a partial pressure of carbon monoxide of 1100 psi or greater and reacting the reaction mixture at a temperature of 90° C. or greater under conditions such that the reaction mixture is and remains substantially saturated with carbon monoxide wherein one or more beta-lactones are formed. The reaction mixture may be subjected to process steps which distribute carbon monoxide throughout the reaction mixture. The reactor utilized for the process may include one or more devices or features which enhance the distribution of carbon monoxide throughout the reaction mixture. The reaction mixture may be intimately mixed such that the carbon monoxide is distributed through the reaction mixture. The disclosed method may be performed such that the selectivity of the reaction toward acetaldehyde or byproducts thereof is 6.0 percent or less. The reaction mixture may be reacted at a temperature of from 90° C. to about 105° C. The reaction mixture may be reacted at a partial pressure of carbon monoxide of greater than about 1200 psi.

The reaction mixture may be reacted in a batch reactor having one or more gas entrainment devices. The mixture may be reacted in a reactor having one or more devices that are adapted to maximize the contact and or distribution of carbon monoxide with or through the reaction mixture. The one or more gas entrainment devices may comprise one or more of, a sparging system to sparge carbon monoxide through the reaction mixture, an entrainment impeller, a gas sparger and the like. The one or more devices that are adapted to maximize the contact of carbon monoxide with the reaction mixture may comprise one or more baffles, or systems which form relatively uniform bubbles which can be utilized to distribute the carbon monoxide throughout the reaction mixture. It is desirable to form bubbles of carbon monoxide wherein the bubbles have high surface areas per unit of volume. The reaction mixture may be reacted in a plug flow reactor having one or more gas entrainment devices. The one or more gas entrainment devices may comprise multiple carbon monoxide injection ports along the plug flow reactor, a gas sparger and the like. The plug flow reactor may comprise one or more devices which promote turbulent flow through the reactor. The one or more devices which promote turbulent flow through the reactor may comprise one or more baffles and the like.

The epoxide may have at least one hydrogen and the beta-lactone may have a beta-hydrogen. The epoxide may correspond to the formula

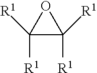

and the beta-lactone may correspond to the formula

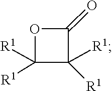

wherein R¹ is independently in each occurrence hydrogen, a hydrocarbyl moiety or a fluorocarbyl moiety; the hydrocarbyl or fluorocarbyl moieties may optionally contain at least one heteroatom or at least one substituent, with the proviso that one of the R¹ on the beta carbon atom is hydrogen. The epoxide may be ethylene oxide, propylene oxide or combination thereof and the beta-lactone may be propiolactone or methyl beta propiolactone, or combinations thereof. The catalyst may be metal carbonyl catalyst. The metal carbonyl catalyst may be represented by [QMy(CO)w]x where: Q is any ligand; M is a metal atom; y is an integer from 1 to 6 inclusive; w is a number that renders the metal carbonyl stable; x is an integer from −3 to +3 inclusive.

The disclosed method may further comprise a second gas. The second gas may be an inert gas, hydrogen, nitrogen or mixture thereof. The method may be performed wherein the epoxide and catalyst are present in amounts such that the epoxide and catalyst have a molar ratio of epoxide/catalyst of about 1500:1 or greater. The method may be performed wherein the solvent is an ether, hydrocarbon, aprotic polar solvent or mixture thereof. The method may be performed in a continuously stirred reactor and the average residence time may be about 5 minutes to 240 minutes. The method may be performed in a plug flow reactor. The plug flow reactor may be a vertical plug flow reactor.

The method disclosed provides an efficient process with high turnover levels and low impurity levels. The turnover number may be 5,000 or greater, 8,000 or greater, 11,000 or greater or 12,000 or greater. The method may be performed such that water is present in the reaction mixture at a concentration of 150 parts per million or less. The formed beta-lactones may be used to prepare other organic compounds or to prepare polymers which are compostable. The starting ingredients may be partially or completely sourced from biologically based feedstocks thus providing for an environmentally friendly life cycle of the final products.

### BRIEF DESCRIPTION OF THE DRAWINGS

FIG. 1 shows an illustration of the hybrid vertical tube reactor useful for the method disclosed herein.

FIG. 2 shows catalyst turnover versus time as it relates to carbon monoxide pressure.

FIG. 3 shows initial EO loading versus Initial catalyst loading at 90° C. for the examples.

### DETAILED DESCRIPTION

While the disclosure has been described in connection with certain embodiments, it is to be understood that the disclosure is not to be limited to the disclosed embodiments and is intended to cover various modifications and equivalent arrangements included within the scope of the appended claims, which scope is to be accorded the broadest interpretation to encompass all such modifications and equivalent structures as is permitted under the law.

One or more means that at least one, or more than one, of the recited components may be used as disclosed. Hydrocarbyl as used herein refers to a group containing one or more carbon atom backbones and hydrogen atoms, which may optionally contain one or more heteroatoms. Where the hydrocarbyl group contains heteroatoms, the heteroatoms may form one or more functional groups well known to one skilled in the art. Hydrocarbyl groups may contain cycloaliphatic, aliphatic, aromatic or any combination of such segments. The aliphatic segments can be straight or branched. The aliphatic and cycloaliphatic segments may include one or more double and/or triple bonds. Included hydrocarbyl groups are alkyl, alkenyl, alkynyl, aryl, cycloalkyl, cycloalkenyl, alkaryl and aralkyl groups. Cycloaliphatic groups may contain both cyclic portions and noncyclic portions. Hydrocarbylene means a hydrocarbyl group or any of the described subsets having more than one valence, such as alkylene, alkenylene, alkynylene, arylene, cycloalkylene, cycloalkenylene, alkarylene and aralkylene. Valence as used herein means a covalent bond between a hydrocarbyl or hydrocarbylene group and another group such as a carbonyl, oxygen, nitrogen or sulfur containing group or atom, or the referenced base compound. As used herein percent by weight or parts by weight refer to, or are based on, the weight of the compositions unless otherwise specified.

Disclosed are methods which comprise the preparation of beta-lactones which provide for shorter reaction times with lower by-product formation. The method comprises contacting carbon monoxide with an epoxide in the presence of a carbonylation catalyst to form a reaction mixture under conditions such that the carbon monoxide is and continues to be distributed throughout the reaction mixture and the reaction mixture is substantially saturated with carbon monoxide. The reaction conditions and reactor designs are chosen to distribute the carbon monoxide throughout the reaction mixture and to maintain the reaction mixture as substantially saturated with carbon monoxide. Under these conditions the formation of beta-lactones over the formation of by-products is favored. Any reaction conditions and reactor designs which favor the formation of beta-lactones over the formation of by-products may be utilized. The carbon monoxide is contacted with one or more epoxides in one or more liquid solvents in the presence of one or more carbonylation catalysts to form a reaction mixture. The formed reaction mixture is exposed to reaction conditions such that beta-lactones are formed with minimized by-product formation. The disclosed method allows for the use of higher reaction temperatures while minimizing the formation of by-products. The use of higher temperatures reduces reaction times and enhances higher efficiency of the method.

Carbon monoxide is a gas that is fed to the reaction mixture in the gaseous state. Any known source if carbon monoxide may be utilized, including carbon monoxide from biobased feedstocks or fossil-based feedstocks. Carbon monoxide may be the only gas present or may be mixed with or entrained in another gas. Carbon monoxide may be mixed with hydrogen such as in a commercial syngas.

The epoxide may be any epoxide that when contacted with carbon monoxide in the presence of a carbonylation catalyst will form a beta-lactone. The epoxide used in the carbonylation reaction may be any cyclic alkoxide containing at least two carbon atoms and one oxygen atom. The epoxide may correspond to the formula

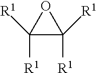

wherein R¹ is independently in each occurrence hydrogen, a hydrocarbyl moiety or a fluorocarbyl moiety; the hydrocarbyl or fluorocarbyl moieties may optionally contain at least one heteroatom or at least one substituent, with the proviso that one of the R¹ on the beta carbon atom is hydrogen. R¹ may be, independently is each occurrence, hydrogen, a halogen substituted alkyl group, a sulfonic acid substituted alkyloxy group; an alkyl sulfonate alkyloxy group; alkyl ether substituted alkyl group; a polyalkylene oxide substituted alkyl group; an alkyl ester substituted alkyl group; an alkenyloxy substituted alkyl group; an aryl ester substituted alkyl group; an alkenyl group; a cyano substituted alkyl group; an alkenyl ester substituted alkyl group; a cycloalkyl substituted alkyl group; an aryl group; a heteroatom containing cycloalkenyl, alkyl ether substituted alkyl group; a hydroxyl substituted alkyl group; a cycloaliphatic substituted alkenyl group; an aryl substituted alkyl group; a haloaryl substituted alkyl group; an aryloxy substituted alkyl group; an alkyl ether substituted alkaryl group; a hetero atom containing cycloaliphatic group substituted alkyl group; a hetero atom containing aryl substituted alkyl group; an alkyl amide substituted alkyl group; an alkenyl substituted cycloaliphatic group; two R¹ may form a cyclic ring, which may optionally contain one or more unsaturated groups; an alkyl group substituted with a beta propiolactone group which may optionally contain one or more ether groups and/or one or more hydroxyl groups; a glycidyl ether group, or a benzocyclobutene substituted alkyl group, optionally substituted with one or more ether groups; with the proviso that one of the R¹ on the beta carbon atom is hydrogen. R¹ may be independently selected from: hydrogen; C₁-C₁₅ alkyl groups; halogenated alkyl chains; phenyl groups; optionally substituted aliphatic or aromatic alkyl groups; optionally substituted phenyl; optionally substituted heteroaliphatic alkyl groups; optionally substituted 3 to 6 membered carbocycle; and optionally substituted 3 to 6 membered heterocycle groups, where two of R¹ may be optionally be taken together with intervening atoms to form a 3 to 10 membered, substituted or unsubstituted ring optionally containing one or more hetero atoms; or any combination thereof. All of R¹ may be hydrogen. Exemplary starting epoxides may be ethylene oxide, propylene oxides, butylene oxides, and the like. The starting epoxide may be ethylene oxide and/or propylene oxide. The starting epoxide may be ethylene oxide.

The beta lactones prepared by the method disclosed include any lactone that may be prepared from the epoxides described. The beta lactones may correspond to the formula;

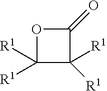

wherein R¹ may be as described hereinbefore. The beta lactone may be beta propiolactone or methyl beta-propiolactone. The beta lactone may be beta propiolactone.

The solvent may be any solvent that facilitates the disclosed method proceeding as described. The solvent may be polar. The solvent may be aprotic. The solvent may be polar aprotic. The solvent may be a hydrocarbon, ketone, acetone, alkyl acetate, pyrrolidone, nitrile, imidazolidinone, halogenated hydrocarbon, carbonate, thioether, dibasic ester or ether. The solvent may be an ether, hydrocarbon, aprotic polar solvent or mixture thereof. The solvent may be tetrahydrofuran, 2,5-dimethyl tetrahydrofuran, sulfolane, N-methyl pyrrolidone, 1,3 dimethyl-2-imidazolidinone, diglyme, triglyme, tetraglyme, diethylene glycol dibutyl ether, isosorbide ethers, methyl tertbutyl ether, diethylether, diphenyl ether, 1,4-dioxane, ethylene carbonate, propylene carbonate, butylene carbonate, dibasic esters, diethyl ether, acetonitrile, ethyl acetate, propyl acetate, butyl acetate, 2-butanone, cyclohexanone, toluene, difluorobenzene, dimethoxy ethane, acetone, methylethyl ketone, or mixture thereof. The solvent may be tetrahydrofuran.

The carbonylation catalyst as described herein functions to catalyze a reaction of an epoxide and carbon monoxide to produce one or more propiolactones and other products. The carbonylation catalyst includes at least a metal carbonyl that is anionic and a Lewis acid that is cationic.

The metal carbonyl of the carbonylation catalyst functions to provide the anionic component of the carbonylation catalyst. The carbonylation catalyst may include one or more, two more, or a mixture of metal carbonyls. The metal carbonyl may be capable of ring-opening an epoxide and facilitating the insertion of CO into the resulting metal carbon bond. In some examples, the metal carbonyl may include an anionic metal carbonyl moiety. In other examples, the metal carbonyl compound may include a neutral metal carbonyl compound. The metal carbonyl may include a metal carbonyl hydride or a hydrido metal carbonyl compound. The metal carbonyl may be a pre-catalyst which reacts in situ with one or more reaction components to provide an active species different from the compound initially provided. The metal carbonyl includes an anionic metal carbonyl species. in some examples, the metal carbonyl may have the general formula [Q_{d}M′ₑ(CO)_{w}]^{y+}, where Q is an optional ligand, M′ is a metal atom, d is an integer between 0 and 8 inclusive, e is an integer between 1 and 6 inclusive, w is a number such as to provide the stable anionic metal carbonyl complex, and y is the charge of the anionic metal carbonyl species. The metal carbonyl may include monoanionic carbonyl complexes of metals from groups 5, 7 or 9 of the periodic table or dianionic carbonyl complexes of metals from groups 4 or 8 of the periodic table. The metal carbonyl may contain cobalt, manganese, ruthenium, or rhodium. Exemplary metal carbonyls may include [Co(CO)₄]⁻, [Ti(CO)e]²⁻, [V(CO)₆]⁻, [Rh(CO)₄]⁻, [Fe(CO)₄]²⁻, [Ru(CO)₄]²⁻, [Os(CO)₄]²⁻, [Cr₂(CO)₁₀]²⁻, [Fe₂(CO)₈]²⁻, [Tc(CO)₅]⁻, [Re(CO)₅]⁻, and [Mn(CO)₅]⁻. The metal carbonyl may be a mixture of two or more anionic metal carbonyl complexes in the carbonylation catalysts used in the methods.

A metal carbonyl additive functions to deliver a metal carbonyl to a Lewis acid that is suitable to combine and form the carbonylation catalyst. The metal carbonyl additive may function to decouple a halogen or a polymer containing a residue of a propiolactone, an epoxide, or both from a metal centered compound to form the carbonylation catalyst that includes the Lewis acid and metal carbonyl combination. The metal carbonyl additive includes at least a metal carbonyl as described herein and a cationic compound. The cationic compound may include lithium, sodium, potassium, rubidium, cesium, beryllium, magnesium, calcium, strontium, barium, radium, or any combination thereof. The metal carbonyl additive may be a salt. The metal carbonyl additive may be a silicon salt in the form of R₃Si—, where R is independently selected from a phenyl, halophenyl, hydrogen, alkyl, alkylhalo, alkoxy, or any combination thereof.

The Lewis acid functions to provide the cationic component of the carbonylation catalyst. The Lewis acid may be a metal centered compound, a metal complex, or both that is configured to be anionically balanced by one or more metal carbonyls. The Lewis acid component of the carbonylation catalyst may include a dianionic tetradentate ligand. The Lewis acid may include one or more porphyrin derivatives, salen derivatives, dibenzotetramethyltetraaza [14]annulene (tmtaa) derivatives, phthalocyaninate derivatives, derivatives of the Trost ligand, tetraphenylporphyrin derivatives, tetramethyl-tetra-aza-annulene type, and corrole derivatives. In some examples, where the carbonylation catalysts used in the disclosed methods include a cationic Lewis acid including a metal complex, the metal complex has the formula [(L^{c})ᵥM_{b}]^{z+},
where:
L is a ligand where, when two or more L are present, each may be the same or different;
M is a metal atom where, when two M are present, each may be the same or different;
v is an integer from 1 to 4 inclusive;
b is an integer from 1 to 2 inclusive; and
z is an integer greater than 0 that represents the cationic charge on the metal complex.

In other examples, the Lewis acid or metal centered compound may have a structure of metal complex I or II. Where the Lewis acid has the metal complex I, the metal complex may be the following configuration:

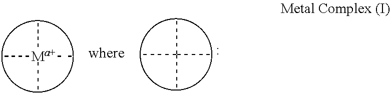

is a multidentate ligand;
M is a metal atom coordinated to the multidentate ligand; and
a is the charge of the metal atom and ranges from 0 to 2. In some examples, the metal complexes include structures according to metal complex II.

In other examples, the Lewis acid may have the metal complex having the formula of metal complex II:

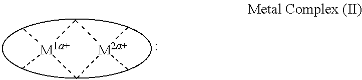

Where a is as defined above and each a may be the same or different,
M1 is a first metal atom;
M2 is a second metal atom; and

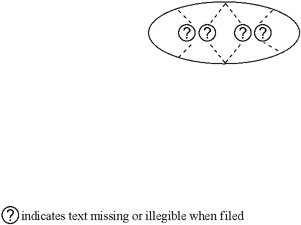

comprises a multidentate ligand system capable of coordinating both metal atoms.

As stated above, the Lewis acid may include or be one or more of porphyrin derivatives (ligand structure 1), salen derivatives (ligand structure 2), dibenzotetramethyltetraaza [14]annulene (tmtaa) derivatives (ligand structure 3), phthalocyaninate derivatives (ligand structure 4), derivatives of the Trost ligand (ligand structure 5), tetraphenylporphyrin derivatives (ligand structure 6), and corrole derivatives (ligand structure 7). The configurations of each of the ligand structures is shown and described below:

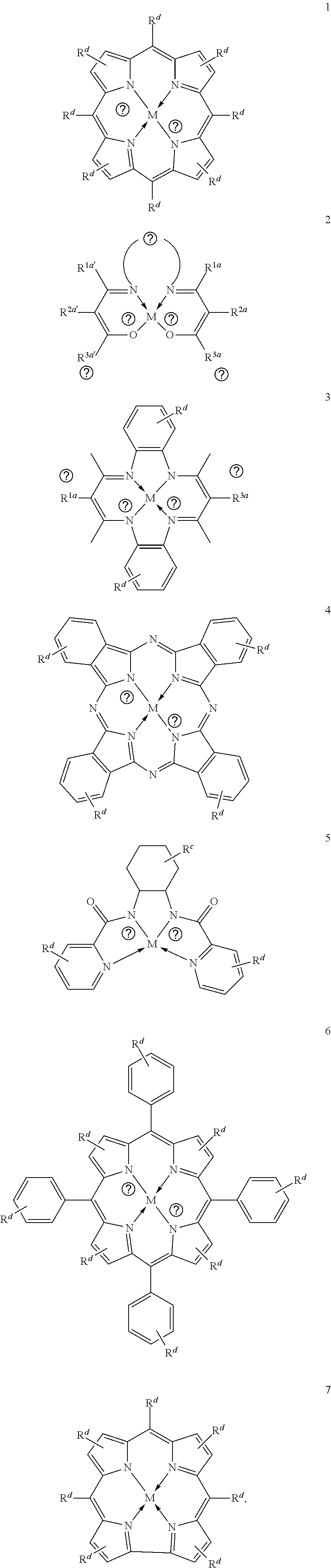

where M is a metal;
where each ligand structure has an ionic charge of 0 to +4;
where R^{1a}, R^{1a′}, R^{2a}, R^{2a′}, R^{3a}, R^{3a′}, R^{d}, and R^{c} at each occurrence is independently hydrogen, halogen, —OR⁴, —NR^{y}₂, —SR, —CN, —NO₂, —SO₂R^{y}, —SOR^{y}, —SO₂NR^{y}₂; —CNO, —NRSO₂R^{y}, —NCO, —N₃, —SiR₃; or an optionally substituted group selected from the group consisting of C₁₋₂₀ aliphatic; C₁₋₂₀ heteroaliphatic having 1-4 heteroatoms independently selected from the group consisting of nitrogen, oxygen, and sulfur; 6 to 10 membered aryl; 5 to 10 membered heteroaryl having 1-4 heteroatoms independently selected from nitrogen, oxygen, and sulfur; and 4 to 7 membered heterocyclic having 1-2 heteroatoms independently selected from the group consisting of nitrogen, oxygen, and sulfur, where two or more R^{d} groups may be taken together to form one or more optionally substituted rings;
where each R^{y} is independently hydrogen, an optionally substituted group selected the group consisting of acyl; carbamoyl, arylalkyl; 6 to 10 membered aryl; C₁₋₁₂ aliphatic; C₁₋₁₂ heteroaliphatic having 1-2 heteroatoms independently selected from the group consisting of nitrogen, oxygen, and sulfur; 5 to 10 membered heteroaryl having 1-4 heteroatoms independently selected from the group consisting of nitrogen, oxygen, and sulfur; 4 to 7 membered heterocyclic having 1-2 heteroatoms independently selected from the group consisting of nitrogen, oxygen, and sulfur; an oxygen protecting group; and a nitrogen protecting group; two R^{y} on the same nitrogen atom are taken with the nitrogen atom to form an optionally substituted 4 to 7 membered heterocyclic ring having 0-2 additional heteroatoms independently selected from the group consisting of nitrogen, oxygen, and sulfur;
wherein any of (R^{2a′} and R^{3a′}), (R^{2a} and R^{3a}), (R^{1a} and R^{2a}), and (R^{1a′} and R^{2a′}) may optionally be taken together with the carbon atoms to which they are attached to form one or more rings which may in turn be substituted with one or more R groups;
where each R⁴ independently is a hydroxyl protecting group or R^{y}; and
R^{4a} is selected from the group consisting of:

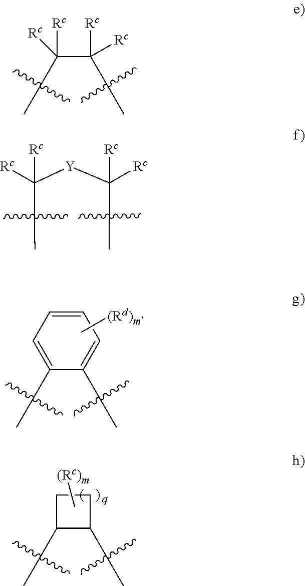

where R^{c} is described above and two or more R^{c} groups may be taken together with the carbon atoms to which they are attached and any intervening atoms to form one or more rings; when two R^{c} groups are attached to the same carbon atom, they may be taken together along with the carbon atom to which they are attached to form a moiety selected from the group consisting of: a 3- to 8-membered spirocyclic ring, a carbonyl, an oxime, a hydrazone, an imine; and an optionally substituted alkene;
Y is a divalent linker selected from the group consisting of: —NR^{y}—, —N(R)C(O)—, —C(O)NR^{y}—, —O—, —C(O)—, —OC(O)—, —C(O)O—, —S—, —SO—, —SO₂—, —C(═S)—, —C(═NR^{y})—, —N═N—; a polyether; a C₃ to Cg substituted or unsubstituted carbocycle; and a C₁₋₈ substituted or unsubstituted heterocycle; m′ is 0 or an integer from 1 to 4;
q is 0 or an integer from 1 to 4, inclusive; and
x is 0, 1, or 2.

In metal complexes 1-2 and/or ligand structures 1-7, M1 and M2 may each independently be a metal atom selected from the periodic table groups 2-13, inclusive. M, M1, M2, or a combination thereof may be a transition metal selected from the periodic table groups 4, 6, 11, 12 and 13. M, M1, M2, or a combination thereof may be aluminum, chromium, titanium, indium, gallium, zinc, cobalt, copper, or any combination thereof. M1 and M2 may be the same or different metals. M1 and M2 may be the same metal but have different oxidation states. M, M1, M2, or a combination thereof may have an oxidation state of +2. M1 or M2 may be Zn(II), Cu(II), Mn(II), Co(II), Ru(II), Fe(II), Co(II), Rh(II), Ni(II), Pd(II) or Mg(II). In certain embodiments M1 is Cu(II). M, M1, M2, or a combination thereof may be Zn(II), Cu(II), Mn(II), Co(II), Ru(II), Fe(II), Co(II), Rh(II), Ni(II), Pd(II) or Mg(II). M, M1, M2, or a combination thereof may have an oxidation state of +3. M, M1, M2, or a combination thereof may be Al(II), Cr(III), Fe(III), Co(III), Ti(III) In(III), Ga(III) or Mn(III). M, M1, M2, or a combination thereof may have an oxidation state of +4. M, M1, M2, or a combination thereof may be Ti(IV) or Cr(IV).

In some Lewis acids, one or more polar ligands may coordinate to M, M1, M2, or a combination thereof and fill the coordination valence of the metal atom. The Lewis acid may include any number of polar ligands to fill the coordination valence of the metal atom. For example, the Lewis acid may include one or more polar ligands, two or more polar ligands, three or more polar ligands, four or more polar ligands, or a plurality of polar ligands. The polar ligand may be a solvent. The polar ligand may be any compound with at least two free valence electrons. The polar ligand may be aprotic. The compound may be tetrahydrofuran, diethyl ether, acetonitrile, carbon disulfide, pyridine, epoxide, ester, lactone, or a combination thereof.

The amount of catalyst may be any useful amount and may depend on the particular reactants and reaction conditions. The useful amount is that amount which catalyzes the reaction to perform at the desired rate of reaction wherein the time of reaction and by product formation are minimized. The amount of catalyst present in the reactor is about 0.001 percent to about 20 percent by weight of the amount of reactants present in the reactor. If the catalyst is a homogeneous catalyst dissolved or entrained in the reaction medium or mixture, the amount of the catalyst may be about 0.001 percent by weight or greater, about 0.01 percent by weight or greater or about 0.05 percent by weight or greater. If the catalyst is a homogeneous catalyst dissolved or entrained in the reaction medium or mixture, the amount of the catalyst may be about 20 precent by weight or less, about 10 precent by weight or less or about 5 precent by weight or less. The amount of homogeneous catalyst when carbonylating an epoxide with carbon monoxide is fed into the reactor along with the epoxide at molar ratio of epoxide to catalyst of about 50:1 or greater or about 100:1 or greater. The molar ratio of epoxide to catalyst may be about 50,000:1 or less, about 25,000 or less, about 10,000 or less, about 5,000 or less, about 2,500 or less or about 2,000 or less.

The catalyst may be heterogeneous wherein the catalyst is anchored to a support. As an illustration, the heterogeneous catalyst may be a supported catalyst useful in the carbonylation of epoxides or lactones such as described in copending application PCT/US2020/044013, published as WO 2021/025918,_incorporated herein by reference. The support may be a porous ceramic such as a packing bead and, may be a zeolite such as described in paragraph 36 of WO 2021/025918, silica, titania, silver (e.g., silver in clay binder). Other exemplary catalysts for carbonylation of epoxides or lactones are described in U.S. Pat. Nos. 6,852,865 and 9,327,280 and U.S. Pat. Appl. Nos. 2005/0014977 and 2007/0213524 each incorporated herein by reference.

The disclosed method is performed with an excess amount of carbon monoxide to epoxide. The method may be performed under conditions such that the reaction medium or mixture is substantially saturated or as close as possible to substantially saturated by carbon monoxide. The reaction may be performed under conditions to distribute the carbon monoxide throughout the reaction medium or mixture. The use of a reaction medium or mixture which is substantially saturated may result in faster reaction times and lower by-product formation. Good distribution of the carbon monoxide through the reaction medium or mixture may result in faster reaction times and lower by-product formation. Performing the method as described allows for faster reaction times and lower by-product formation by favoring formation of the desired product. The molar ratio of carbon monoxide to epoxide may be any ratio that allows rapid reactions and low by-product formation. The molar ratio of carbon monoxide to epoxide may be greater than 1:1, 1.1:1 or greater, 1.2:1 or greater, 1.4:1 or greater or about 1.5:or greater. The molar ratio of carbon monoxide to epoxide may be about 20:1 or less, about 10:1 or less, about 7:1 or less, about 5:1 or less, about 4:1 or less, about 3:1 or less or about 2:1 or less. Substantially saturated means that the reaction medium or mixture contains the amount of carbon monoxide at or near the amount to fully saturate the reaction mixture or medium. Substantially saturated means the rate of introduction of carbon monoxide throughout the reaction medium is faster than the rate of formation of acetaldehyde or byproducts thereof. The amount needed to saturate the reaction mixture or medium may vary based on the choice of solvent, reaction temperatures and any pressure in the headspace of the reactor by carbon monoxide and any other gas present. Substantially saturated may mean that the reaction mixture contains 95 percent by weight or greater of the carbon monoxide of a fully saturated reaction mixture under the reaction conditions, the reaction mixture contains 98 percent by weight or greater of the carbon monoxide of a fully saturated reaction mixture or contains 99 percent by weight or greater of the carbon monoxide of a fully saturated reaction mixture.

The method may be performed at temperatures at which the rate of reaction is faster than conventional disclosed processes. The method may be performed at temperatures of 90° C. or greater or about 100° C. or greater. The method may be performed at temperatures of about 110° C. or less or about 105° C. or less.

The method may be performed under pressure provided by the presence of gas in the reactor. The gas may be carbon monoxide or carbon monoxide and a second gas. The second gas may be a gas which does not impact or participate in the reaction. The second gas may be an inert gas. The second gas may be nitrogen, hydrogen, argon, and the like. The partial pressure of carbon monoxide may be any pressure that facilitates performing the method at higher rates with the formation of lower amounts of by-products. The partial pressure of carbon monoxide may be 900 psi or greater, about 1100 psi, 1200 psi or greater, 1300 psi or greater or about 1500 psi or greater. The partial pressure of carbon monoxide may be about 2000 psi or less, about 1800 psi or less or about 1600 psi or less.

The time period for performing the method may be any time period that facilitates high conversion to beta lactones. The reaction time may be dependent on the reactor type and process conditions. The method may be performed in a batch process or may be performed in a semicontinuous or a continuous process. The reaction time for purposes of this discussion is the residence time of the reactants in the reactor, regardless of the process type. The residence time of the reactants in the reactor may be about 5 minutes or greater, about 10 minutes or greater, 15 minutes or greater or about 20 minutes or greater. The residence time of the reactants in the reactor may be about 240 minutes or less, about 180 minutes or less or about 60 minutes or less.

The method may be performed in a reactor which is equipped with one or more devices that entrain gases, such as carbon monoxide, in the reaction medium or mixture. As used herein entraining a gas in the reaction medium means to dissolve or disperse the gas in the reaction medium so as to facilitate the reaction of carbon monoxide with the epoxides as described herein. Any device that creates turbulent flow in the reactor, increases the amount of gas contacting the reaction mixture, intimately mixes the carbon monoxide with the reaction medium or facilitates forming a mixture that is substantially saturated with carbon monoxide may be used. Exemplary entrainment devices include gas sparging devices, mixing systems which intimately mix the reaction medium or mixture, one or more gas injection ports, baffles in the reactor, and the like.

Reactors which may be utilized in batch or semi-continuous reaction modes include continuously stirred reactors, and the like. Continuous reactors include plug flow reactors, bubble column reactors and buss loop type reactors and the like. Entrainment devices that are useful in the processes include Ruston impellers, hollow shaft impellers and blade impellers and the like. The reaction mixture may be reacted in a plug flow reactor having one or more gas entrainment devices. Such entrainment devices may be any devices which create turbulent flow thorough the plug flow reactor, for example baffles located in the reactor. Plug flow reactors may be equipped with multiple gas injection ports, a gas sparger, and the like. The plug flow reactor may be a vertical or horizontal plug flow reactor. In reactors useful for batch or semi-continuous processes the gas entrainment devices may be mixing systems, sparging systems, baffles in the reactor, and the like. Mixing systems may include impellers, and the like. Any reactor may have one or more of the disclosed gas entrainment devices. The reactors may contain a packing to promote better contact of the carbon monoxide with the catalyst/liquid.

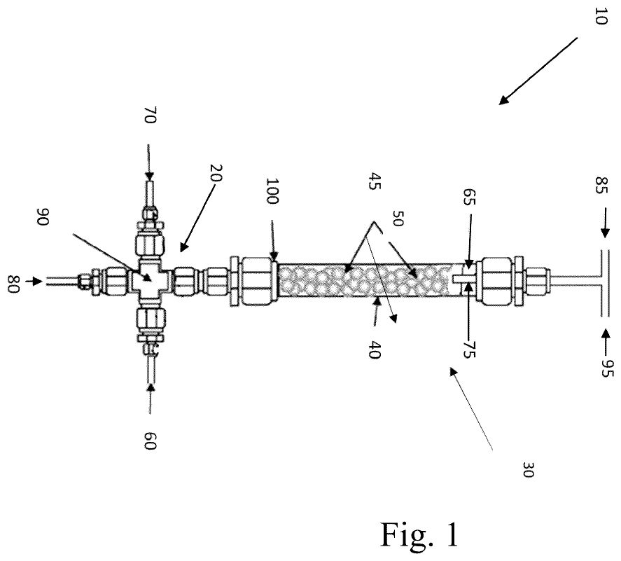

An exemplary method is described hereinafter. The method employs a hybrid vertical bubble plug flow reactor (reactor) 10 illustrated in FIG. 1. The reactor 10 has a bottom inlet 20 and top outlet 30 connected by tubular member 40. The bottom inlet is comprised of separate gas reactant inlet 60 and liquid reactant inlet 70. The bottom inlet may be further comprised of a drain 80 or other inlet for introduction of other components. In the bottom inlet 20 the gas reactant is mixed with the liquid reactant which may be mixed with a solvent in mixing area 90. The gas reactant inlet 60 may also be comprised of a sparger (not shown) to cause the formation of desired gas reactant bubble size within the liquid reactant and to facilitate the saturation of the liquid reactant and, if used, solvent. The inlet may further be comprised of a screen mesh or the like 100 or the like to retain optional packing 45 in the vertical tubular member 40 or to further disperse or reduce the gas reactant bubbles. The mesh may be any to be used to encourage the rupture of bubbles into smaller bubbles and retain packing such as the product offered from Sealing Devices Inc., a pipe flange gasket with inlaid 316SS #20 mesh. The bottom inlet may be configured in any manner that allows for the injection and mixture of the reactants at the bottom of the tubular member 40 such as radial inlets at the bottom of the tubular member 40 or vertical tubes traversing within the tubular member from the top and exiting at the bottom of tubular member 40. The outlet 30 is fitted with any suitable gas liquid separation method such as those known in the art. The outlet 30 may be comprised of an extraction tube 75 that extends sufficiently into the liquid product, solvent and residual liquid reactant (reactor liquid 50) to allow for the extraction of said reactor liquid 50 and to allow head space 65 to be maintained providing an overpressure of the gas reactant. In an alternative, the extraction tube may be replaced by any other outlet such as a radial extraction port that also allows the headspace to be maintained. The extraction tube, port or other outlet for the liquid product and residual solvent and unreacted liquid reactants, may be accompanied by a separate gas outlet. The outlet 30 has effluent outlet 95 for transporting the reactor liquid 50 for further processing such as separation, further reaction, recovery of solvent and/or catalyst and gaseous outlet 85 for maintaining the overpressure of gases within the reactor utilizing suitable flow controllers, tanks, valves and apparatus such as those known in the art, which may be integrated or integral with the reactor.

In performing the method, a liquid reactant is injected into the mixing area 90 through the liquid reactant inlet 70 and the gas reactant is injected into the mixing area 90 through the gas reactant inlet 60 where bubbles of the gas reactant are formed in the liquid reactant. The reactants may be cooled or heated when injected depending on the type of reaction being performed. For example, it may be desirable to inject cooled reactants for exothermic reactions such as carbonylation described herein. The tubular member 40 may be vertically oriented with it being understood some deviation from vertical may be acceptable, but, in essence, the reaction zone is straight without any bends or other obstructions that can entrap the gas reactant. The tubular member 40 may have any cross-sectional shape such a square, rectangle, quadrilateral, hexagon, pentagon oval or circle with a circle being preferred. The materials of construction may be any that is compatible with the reactants and conditions used to react the reactants and is readily determinable by one of ordinary skill in the art. For example, when the reactants are an epoxide and carbon monoxide, stainless steel (e.g., 302 or 316 stainless steel), inorganic glasses, organic plastics (e.g. engineering polymers) and ceramics may be used. The length of the reactor 10 and tubular member 40 may be any length and diameter useful to realize the desired reaction conditions such as residence time. Typically, the diameter of the tubular member may be from 2 mm, 3 mm, 5 mm or 1 cm to 200 cm, 100 cm, 50 cm, 20 cm or 10 cm. The diameter of a non-cylindrical tubular member is taken as the largest dimension of the cross-section of such tubular member. The aspect ratio (length/diameter) may be at least about 10, 15 or 20 to any commercially practicable ratio such as 1000, 500, 200, 100, 75 or 50. The reactor 10 may be comprised of multiple tubular members 40 in parallel with separate or shared flanging for injection of the reactants and removal of the products. Such parallel configurations of tubular members 40 may be contained in a common vessel, for example, that may have a heating element or heat transfer fluid to heat or cool such tubular members 40 commonly. The reactors 10 may be configured in series, for example, to inject further or different reactants or catalysts or react the products from one reactor in a subsequent reactor to form a different product.

The tubular member 40 may be further comprised of one or more radial inlets along its length for injecting further reactants or other components (e.g., solvent, stabilizers, surfactants or the like). Other components, just like the solvent may also be injected in the bottom inlet. A radial inlet may be used to inject the same gas reactant or liquid reactant inserted in the bottom inlet 20 along the length of the tubular member 40. If a gas reactant is injected through a radial inlet it may incorporate a sparger as described herein. Differing reactants may be inserted through the radial inlet or inlets to form differing desired final products. The reactants or any further components may be heated or cooled depending on the desired reaction or reaction conditions.

The methods as disclosed may be performed under conditions such that high turnover numbers are achieved. the method allows for the catalyst to be more effectively and efficiently used realizing high TONs. Turnover Number (TON) is used as commonly understood in the art, wherein for continuous reactions the amount of catalyst and product produced in a given time results in the TON for continuous reactions and is given by (moles product/time)/(moles catalyst/time). TON indicates the efficacy of the catalyst for continuous reactions where the output of the product is similar. TON in batch processes is determined based on (moles product)/(moles catalyst). The turnover number may be about 5,000 or greater, about 8,000 or greater, about 11,000 or greater or about 12,000 or greater.

The method may be performed such that the percent selectivity of the reaction to acetaldehyde or a byproduct formed from acetaldehydeis is about 6.0 or less, 4.0 or less, 3.0 or less or about 2.0 or less. Selectivity to acetaldehyde or a byproduct formed from acetaldehyde is determined according to the formula,

Selectivity_{ACH}%=(grams of ACH produced/grams of EO added)*100.

All patent and literature references disclosed herein are incorporated in their entirety for all purposes.

### EMBODIMENTS

The following are embodiments of the disclosed compositions and methods.

1. A method comprising contacting carbon monoxide with one or more epoxides in one or more liquid solvents in the presence of one or more carbonylation catalysts to form a reaction mixture in the liquid solvents, feeding gaseous carbon monoxide to the reaction mixture such that the reaction mixture is under a partial pressure of carbon monoxide of 1100 psi or greater and reacting the reaction mixture at a temperature of 90° C. or greater under conditions such that the reaction mixture is and remains substantially saturated with carbon monoxide wherein one or more beta-lactones are formed.

2. A method according to Embodiment 1 wherein the reaction mixture is mixed and the carbon monoxide is distributed through the reaction mixture.

3. A method according to Embodiment 1 or 2 wherein an effluent containing the reaction mixture and the one or more beta lactones formed is recovered from the method wherein the effluent and the one or more beta lactones formed exhibits a Selectivity ACH of 6.0 percent or less wherein Selectivity_{ACH}%=(grams of ACH produced/grams of EO added)*100%.

4. A method according to any of the preceding Embodiments wherein an effluent containing the reaction mixture and the one or more beta lactones formed are recovered from the method wherein the effluent and the one or more beta lactones formed exhibits a Selectivity ACH of 4.0 percent or less

5. A method according to any of the preceding Embodiments wherein the reaction mixture is reacted at a temperature of from 90° C. to about 105° C.

6. A method according to any of the preceding Embodiments wherein the reaction mixture is reacted at a partial pressure of carbon monoxide of greater than 1200 psi.

7. A method according to any of the preceding Embodiments wherein the reaction mixture is reacted at a partial pressure of carbon monoxide of about 1500 psi or greater.

8. A method according to any of the preceding Embodiments wherein the reaction mixture is reacted at partial pressure of carbon monoxide of about 1500 psi to about 2000 psi.

9. A method according to any of the preceding Embodiments wherein the reaction mixture is reacted in a batch reactor having one or more gas entrainment devices.

10. A method according to any of the preceding Embodiments wherein the mixture is reacted in a reactor having one or more devices that are adapted to maximize the contact of carbon monoxide with the reaction mixture.

11. A method according to any of the preceding Embodiments wherein the one or more gas entrainment devices comprise, a sparging system to sparge carbon monoxide through the reaction mixture, an entrainment impeller, a gas sparger, a Ruston impeller, hollow shaft impeller and blade impeller.

12. A method according to any of the preceding Embodiments wherein the one or more devices that are adapted to maximize the contact of carbon monoxide with the reaction mixture comprise one or more baffles.

13. A method according to any of Embodiments 1 to 8 wherein the reaction mixture is reacted in a plug flow reactor having one or more gas entrainment devices.

14. A method according to Embodiment 13 wherein the one or more gas entrainment devices comprise multiple carbon monoxide injection ports along the plug flow reactor, a gas sparger, Ruston impeller, hollow shaft impeller and blade impeller.

15. A method according to Embodiment 13 or 14 wherein the plug flow reactor comprises one or more devices which promote turbulent flow through the reactor.

16. A method according to Embodiment 15 wherein one or more devices which promote turbulent flow through the reactor comprise one or more baffles.

17. A method according to any of the preceding Embodiments wherein the epoxide has at least one hydrogen and the beta-lactone has a beta-hydrogen.

18. A method according to any of the preceding Embodiments wherein the epoxide corresponds to the formula

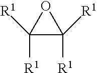

and the beta-lactone corresponds to the formula

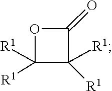

wherein R1 is independently in each occurrence hydrogen, a hydrocarbyl moiety or a fluorocarbyl moiety; the hydrocarbyl or fluorocarbyl moieties may optionally contain at least one heteroatom or at least one substituent, with the proviso that one of the R1 on the beta carbon atom is hydrogen.

19. A method according to Embodiment 18 wherein R¹ is hydrogen, a halogen substituted alkyl group, a sulfonic acid substituted alkyloxy group; an alkyl sulfonate alkyloxy group; alkyl ether substituted alkyl group; a polyalkylene oxide substituted alkyl group, an alkyl ester substituted alkyl group; an alkenyloxy substituted alkyl group; an aryl ester substituted alkyl group; an alkenyl group; a cyano substituted alkyl group; an alkenyl ester substituted alkyl group; a cycloalkyl substituted alkyl group; an aryl group; a heteroatom containing cycloalkenyl, alkyl ether substituted alkyl group; a hydroxyl substituted alkyl group, a cycloaliphatic substituted alkenyl group; an aryl substituted alkyl group; a haloaryl substituted alkyl group; an aryloxy substituted alkyl group; an alkyl ether substituted alkaryl group; a hetero atom containing cycloaliphatic group substituted alkyl group; a hetero atom containing aryl substituted alkyl group, an alkyl amide substituted alkyl group, an alkenyl substituted cycloaliphatic group; two R¹ may form a cyclic ring, which may optionally contain one or more unsaturated groups; an alkyl group substituted with a beta propiolactone group which may optionally be contain one or more ether groups and/or one or more hydroxyl groups; a glycidyl ether group, or a benzocyclobutene substituted alkyl group, optionally substituted with one or more ether groups; with the proviso that one of the R1 on the beta carbon atom is hydrogen.

20. A method according to Embodiment 18 wherein all R¹ are hydrogen.

21. A method according to any one of the preceding Embodiments, wherein the epoxide is ethylene oxide, propylene oxide or combination thereof and the beta-lactone is propiolactone or methyl beta propiolactone, or combinations thereof.

22. A method according to any one of the preceding Embodiments, wherein the epoxide is ethylene oxide and the beta-lactone is beta propiolactone.

23. The method of any one of the preceding Embodiments further comprising a second gas.

24. The method of Embodiment 23, wherein the second gas is an inert gas, argon, nitrogen or mixture thereof.

25. The method of any of the preceding Embodiments, wherein the epoxide and catalyst are present in amounts such that the epoxide and catalyst have a molar ratio of epoxide/catalyst of greater than 1500.

26. The method of Embodiment 25, wherein the epoxide/catalyst molar ratio is 2,000 to 25,000.

27. The method of any one of the preceding Embodiments, wherein the catalyst is a homogeneous catalyst.

28. The method of Embodiment 27, wherein the catalyst is metal carbonyl catalyst.

29. The method of any one of the preceding Embodiments, wherein the metal carbonyl catalyst is represented by [QMy(CO)w]x where: Q is any ligand; M is a metal atom; y is an integer from 1 to 6 inclusive; w is a number that renders the metal carbonyl stable; x is an integer from −3 to +3 inclusive.

30. The method of Embodiment 29, wherein M is Ti, Cr, Mn, Fe, Ru, Co, Rh, Ni, Pd, Cu, Zn, Al, Ga or In.

31. The method of Embodiment 29, wherein M is Co.

32. The method of any one of Embodiments 27 to 31, wherein the metal carbonyl catalyst is anionic and further comprised of a cationic Lewis acid.

33. The method of Embodiment 32, wherein the cationic Lewis acid is a metal complex represented by [M′(L)b]c+ wherein, M′ is a metal; each L is a ligand; b is an integer of 1 to 6; c is 1, 2, or 3; and if more than one L is present, each L may be the same or different.

34. The method of Embodiment 33, wherein the ligand L is a dianionic tetradentate ligand.

35. The method of Embodiment 34, wherein the dianionic tetradentate ligand is a porphyrin derivative, salen derivative, dibenzotetramethyltetraaza 14 annulene derivative; phthalocyaninate derivative, derivative of the Trost ligand or combination thereof.

36. The method of Embodiment 35, wherein the dianionic tetradentate ligand is a porphyrin derivative.

37. The method of any one of Embodiments 33 to 36, wherein M′ is a translation metal or group 13 metal.

38. The method of any one of 33 to 37, wherein M′ is aluminum, chromium, indium, gallium or combination thereof.

39. The method of Embodiment 38, wherein M′ is aluminum, chromium or combination thereof.

40. The method of Embodiment 38 or 39, wherein M′ is aluminum, chromium or combination thereof.

41. The method of any one of the preceding Embodiments wherein the solvent is an ether, hydrocarbon, aprotic polar solvent or mixture thereof.

42. The method of Embodiment 41, wherein the solvent is tetrahydrofuran, 2,5-dimethyl tetrahydrofuran, sulfolane, N-methyl pyrrolidone, 1,3 dimethyl-2-imidazolidinone, diglyme, triglyme, tetraglyme, diethylene glycol dibutyl ether, isosorbide ethers, methyl tertbutyl ether, diethylether, diphenyl ether, 1,4-dioxane, ethylene carbonate, propylene carbonate, butylene carbonate, dibasic esters, diethyl ether, acetonitrile, ethyl acetate, propyl acetate, butyl acetate, 2-butanone, cyclohexanone, toluene, difluorobenzene, dimethoxy ethane, acetone, methylethyl ketone, or mixture thereof.

43. The method of Embodiment 42, wherein the solvent is tetrahydrofuran.

44. The method of any one of the preceding Embodiments, wherein the method is performed in a continuously stirred reactor and the average residence time of the reaction mixture is about 5 minutes to about 240 minutes.

45. The method of Embodiment 44, wherein the average residence time of the reaction mixture is about 15 minutes to 120 minutes.

46. The method of any one of the preceding Embodiments, wherein the method is performed in a plug flow reactor.

47. The method of Embodiment 46, wherein the plug flow reactor is a vertical plug flow reactor.

48. The method of any one of the preceding Embodiments, wherein the turnover number is 5,000 or greater.

49. The method of any one of the preceding Embodiments, wherein the turnover number is 11,000 or greater.

50. The method of any one of the preceding Embodiments, wherein water is present in the reaction mixture at a concentration of 150 parts per million or less.

### ILLUSTRATIVE EXAMPLES

The following examples are provided to illustrate the invention but are not intended to limit the scope thereof. All parts and percentages are by weight unless otherwise indicated.

### Test Procedures

Selectivity for the conversion of ethylene oxide to acetaldehyde is compared to the selectivity to the desired beta-propiolactone product in the presence of carbonylation catalyst at various pressures, temperatures, stir rates, reactor configurations, and catalyst loadings.

The process for acetaldehyde selectivity determination involves charging 0.06 mmol of carbonylation catalyst dissolved in 70 mL of dry and degassed tetrahydrofuran with half desired carbon monoxide pressure for experiment into an inert stainless steel reaction vessel affixed with a Mettler Toledo ReactIR sentinel for in-situ reaction progress tracking. Added is ethylene oxide with desired carbon monoxide pressure for experiment. The reaction is kept at a constant pressure of carbon monoxide during the duration of the study. A liquid sample is obtained for GC-TCD once the ethylene oxide has been consumed as indicated by ReactIR.

The process conditions and results are compiled in the following tables, Table 1, Table 2, and Table 3, Table 1 shows the process at 70° C., Table 2 shows the process at 90° C., and Table 3 shows the process at 100° C.

TABLE 1
Experiments compiled at 70°
C., 70 mL THF, and 0.06 mmol catalyst.

| | | | | | dbPL/dt | | |
|---|---|---|---|---|---|---|---|
| | | | | | at 10% | | Selec- |
| | | | PSI | | EO | ACH | tivity |
| Run | RPM | Baffle | CO | EO:CAT | Conv. | Wt % | ACH % |
| 1 | 260 | N | 500 | 1597 | 39 | 0.162 | 2.9 |
| 2 | 260 | N | 500 | 1619 | 43 | 0.220 | 3.3 |
| 3 | 260 | N | 900 | 1485 | 67 | N.D. | 0 |
| 4 | 260 | N | 1200 | 1597 | 75 | N.D. | 0 |
| 5 | 820 | N | 1200 | 1465 | 61 | N.D. | 0 |
| 6 | 880 | Y | 1200 | 1672 | 70 | N.D. | 0 |
| 7 | 516 | Y | 1200 | 1621 | 74 | N.D. | 0 |
| 8 | 640 | Y | 1500 | 1580 | 63 | N.D. | 0 |
| 9 | 640 | Y | 1500 | 1822 | 68 | N.D. | 0 |

Table 1. Experiments compiled at 70° C., 70 mL THF, and 0.06 mmol catalyst.
Grams of acetaldehyde (ACH) determined by GC-TCD weight percent using amount of THF from reaction as a reference.

$${Selectivity}_{ACH} \% = ( grams of ACH produced / grams of EO added ) * 100 \%$$

Table 2. Experiments compiled at 90 EC.
The experiments shown in Table 2 are run at 90° C., using 70 ml of tetrahydrofuran and 0.06 mmol of catalyst.

| | | | | | dbPL/dt | dbPL/dt | | |
|---|---|---|---|---|---|---|---|---|
| | | | | | at 10% | at 25% | | Selec- |
| | | | PSI | | EO | EO | ACH | tivity |
| Run | RPM | Baffle | CO | EO:CAT | Conv. | Conv. | Wt % | ACH % |
| 10 | 260 | N | 500 | 1609 | 112 | 148 | 1.31 | 21.4 |
| 11 | 260 | N | 500 | 1516 | 110 | 120 | — | |
| 12 | 260 | N | 900 | 1510 | 165 | — | 0.54 | 9.4 |
| 13 | 260 | N | 900 | 1488 | 186 | 205 | 0.47 | 8.3 |
| 14 | 820 | N | 900 | 1444 | 171 | 181 | 0.28 | 5.0 |
| 15 | 820 | N | 1200 | 1763 | 171 | 157 | 0.1 | 1.5 |
| 16 | 880 | Y | 1200 | 1546 | 204 | 207 | 0.16 | 2.8 |
| 17 | 890 | Y | 1200 | 1475 | 179 | 218 | 0.14 | 2.5 |
| 18 | 640 | Y | 1200 | 1543 | 215 | 228 | 0.22 | 3.8 |
| 19 | 640 | Y | 1500 | 1561 | 187 | 207 | 0.16 | 2.7 |
| 20 | 260 | Y | 1500 | 1586 | 215 | 216 | 0.22 | 3.6 |
| 21 | 640 | Y | 1500 | 1471 | 231 | 252 | 0.13 | 2.4 |
| 22 | 640 | Y | 1500 | 1582 | 194 | 190 | 0.11 | 1.8 |
| 23 | 640 | Y | 1500 | 1524 | 210 | 213 | 0.12 | 2.1 |

Table 3. Experiments compiled at 100° C.
All reactions completed with a baffle in place. The experiments shown in Table 2 are run at 90° C., using 70 ml of terahydrofuran and 0.06 mmol of catalyst.

| | | | | dbPL/dt | | |
|---|---|---|---|---|---|---|
| | | | | at 25% | | Selec- |
| | | PSI | | EO | ACH | tivity |
| Run | RPM | CO | EO:CAT | Conv. | Wt % | ACH % |
| 24 | 260 | 900 | 1460 | 320 | 1.04 | 18.3 |
| 25 | 260 | 1200 | 1565 | 322 | 0.63 | 10.4 |
| 26 | 260 | 1500 | 2046 | 286 | 0.57 | 7.6 |
| 27 | 630 | 900 | 1430 | 279 | 0.80 | 14.4 |
| 28 | 630 | 1200 | 1488 | 248 | 0.34 | 6.1 |
| 29 | 900 | 1200 | 1433 | | 0.45 | 8.2 |
| 30 | 630 | 1500 | 1376 | 243 | 0.21 | 4.0 |

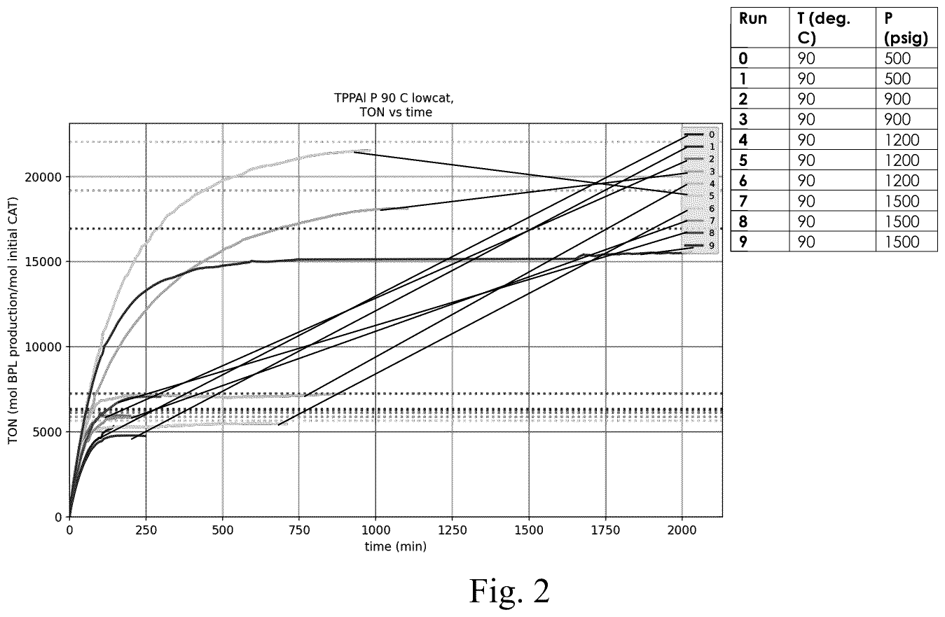

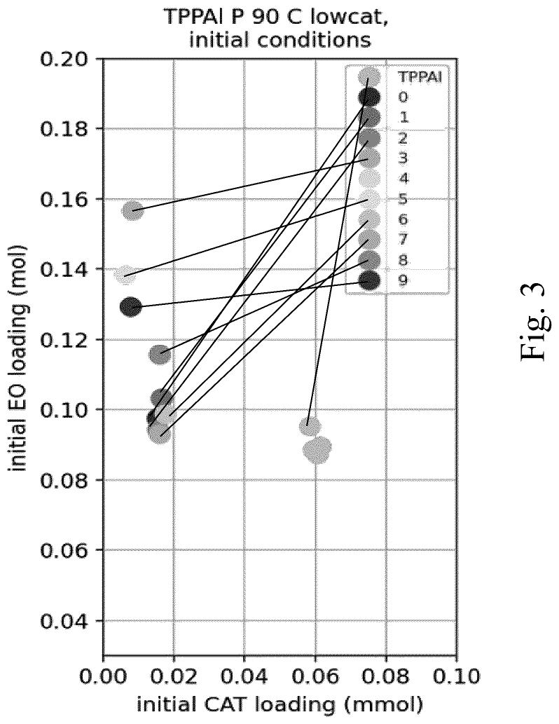

FIG. 2 shows catalyst turnover versus time as it relates to carbon monoxide pressure.
FIG. 3 shows initial EO loading versus Initial catalyst loading at 90 C for the examples,

1. A method comprising contacting carbon monoxide with one or more epoxides in one or more liquid solvents in the presence of one or more carbonylation catalysts to form a reaction mixture in the liquid solvents, feeding gaseous carbon monoxide to the reaction mixture such that the reaction mixture is under a partial pressure of carbon monoxide of 1100 psi or greater and reacting the reaction mixture at a temperature of 90° C. or greater for from about 5 to 240 minutes wherein the reaction mixture is mixed and the carbon monoxide is distributed through the reaction mixture such that the reaction mixture is and remains substantially saturated with carbon monoxide wherein one or more beta-lactones are formed:
wherein the one or more carbonylation catalysts is a cobalt carbonyl that is anionic and a Lewis acid that is cationic and is metal complex represented by [M′(L)b]c+ wherein, M′ is a aluminum, chromium or combination thereof; each L is a ligand; b is an integer of 1 to 6; c is 1, 2, or 3; and if more than one L is present, each L may be the same or different wherein ligand L is a dianionic tetradentate ligand, wherein the dianionic tetradentate ligand is a porphyrin derivative, salen derivative, dibenzotetramethyltetraaza 14 annulene derivative; phthalocyaninate derivative, derivative of the Trost ligand or combination thereof.

2. (canceled)

3. The method according to claim 1, wherein an effluent containing the reaction mixture and the one or more beta lactones formed is recovered from the method wherein the effluent and the one or more beta lactones formed exhibit a Selectivity ACH of 6.0 percent or less wherein Selectivity_{ACH}%=(grams of ACH produced/grams of EO added)*100%.

4. (canceled)

5. The method according to claim 1, wherein the reaction mixture is reacted at a temperature of from 90° C. to about 105° C.

6. A method according to claim 1, wherein the reaction mixture is reacted at a partial pressure of carbon monoxide of greater than 1200 psi.

7-8. (canceled)

9. The method according to claim 1, wherein the reaction mixture is reacted in a batch reactor having one or more gas entrainment devices.

10. The method according to claim 1, wherein the mixture is reacted in a reactor having one or more devices that are adapted to maximize the contact of carbon monoxide with the reaction mixture.

11. The method according to claim 9, wherein the one or more gas entrainment devices comprise, a sparging system to sparge carbon monoxide through the reaction mixture, an entrainment impeller, a gas sparger, a Ruston impeller, a hollow shaft impeller and a blade impellers.

12. (canceled)

13. The method according to claim 1, wherein the reaction mixture is reacted in a plug flow reactor having one or more gas entrainment devices.

14. The method according to claim 13 wherein the one or more gas entrainment devices comprise multiple carbon monoxide injection ports along the plug flow reactor, a gas sparger, Ruston impellers, hollow shaft impellers and blade impellers.

15-16. (canceled)

17. The method according to claim 1, wherein the epoxide has at least one hydrogen and the beta-lactone has a beta-hydrogen.

18-20. (canceled)

21. The method according tom claim 1, wherein the epoxide is ethylene oxide, propylene oxide or combination thereof and the beta-lactone is propiolactone or methyl beta propiolactone, or combinations thereof.

22-35. (canceled)

36. The method of claim 1, wherein the dianionic tetradentate ligand is a porphyrin derivative.

37-40. (canceled)

41. The method of claim 1, wherein the solvent is an ether, hydrocarbon, aprotic polar solvent or mixture thereof.

42-43. (canceled)

44. The method of claim 1, wherein the method is performed in a continuously stirred reactor.

45. The method of claim 44, wherein the average residence time of the reaction mixture is about 15 minutes to about 120 minutes.

46. The method of claim 1, wherein the method is performed in a plug flow reactor.

47. The method of claim 46, wherein the plug flow reactor is a vertical plug flow reactor.

48. The method of claim 1, wherein the turnover number is 5,000 or greater.

49. The method of claim 1, wherein the turnover number is 11,000 or greater.

50. The method of claim 1, wherein water is present in the reaction mixture at a concentration of 150 parts per million or less.
